(19) United States
(12) Patent Application Publication
(10) Pub. No.: US 2016/0346044 A1
(43) Pub. Date: Dec. 1, 2016

(54) FLEXIBLY PLANNED KITTED KNEE PROTOCOL

(71) Applicant: BIOMET MANUFACTURING, LLC, WARSAW, IN (US)

(72) Inventors: David R. Brown, Warsaw, IN (US); Brian Uthgenannt, Winona Lake, IN (US); Robert Metzger, Wakarusa, IN (US)

(21) Appl. No.: 15/167,261

(22) Filed: May 27, 2016

Related U.S. Application Data
(60) Provisional application No. 62/167,591, filed on May 28, 2015.

(51) Int. Cl. A61B 34/10; A61B 90/00; A61B 17/17

(52) U.S. Cl. CPC A61B 34/10; A61B 17/1764; A61B 90/06; A61B 2034/108; A61B 2090/061

(57) ABSTRACT

A method of planning and preparing for a total knee arthroplasty procedure, the method comprising: generating three-dimensional models of a tibia and a femur of a patient; sizing the tibia and the femur to within a range based on the three-dimensional models; selecting a resection tool for each of the tibia and femur based on the three-dimensional models; and packaging the resection tools. A method of planning and preparing for a surgical procedure can comprise: generating a three-dimensional bone model for one or more bones; sizing the one or more bones based on the three-dimensional model; recording a surgical plan based on the three-dimensional bone model; selecting a first surgical tool for the one or more bone based on the surgical plan; and evaluating selection of a second surgical tool based on a performance parameter of the first surgical tool.

### CLAIM OF PRIORITY

This patent application claims the benefit of priority of Brown et al., U.S. Provisional Patent Application Ser. No. 62/167,591, entitled “FLEXIBLY PLANNED KITTED KNEE PROTOCOL,” filed on May 28, 2015, which is hereby incorporated by reference herein in its entirety.

### TECHNICAL FIELD

This document pertains generally, but not by way of limitation, to systems and methods for planning and performing arthroplasty procedures. More particularly, this disclosure relates to, but not by way of limitation, pre-operative planning techniques for selecting and using surgical devices and systems based on pre-operative and intraoperative information.

### BACKGROUND

Arthroplasty procedures for total joint replacement surgeries can involve the use of many different components, such as planning systems, instruments, techniques, procedures and the like. Sometimes there are multiple instruments or techniques that can be used to achieve the same or a similar result. A surgeon has leeway to choose which instruments and techniques to use in any specific surgery based on preference and each particular patient. However, each of these different components do not always lend themselves to compatibility with each other. This often leaves the surgeon to have to make a large number of decisions, each of which may be based upon estimates or assumptions about what will result at each step of the desired procedure. These decisions can increase the length and expense of the pre-planning process and the surgery, and can also be a source for introducing error into the planning process.

Examples of surgical instruments are described in U.S. Pat. No. 8,265,790 to Amiot et al., U.S. Pat. No. 8,591,516 to Metzger et al., and U.S. Pat. No. 8,715,290 to Fisher et al.

### OVERVIEW

The present inventors have recognized, among other things, that a problem to be solved can include the need for surgeons to have to manually plan a surgical procedure by selecting a succession of instruments and techniques that must be performed, without knowing how each selected instrument and technique will affect subsequent decisions.

The present inventors have recognized that the pre-planning and intraoperative planning procedures can be simplified by providing a surgeon with a list of starting points and directing the surgeon through a plurality of optimal subsequent steps based on the previous input.

The present subject matter can help provide a solution to this problem, such as by providing the surgeon with a searchable database that includes a menu of different surgical tools and surgical techniques that can perform different, the same or equivalent results. Thus, the database can include a matrix of surgical tools that are compatible with each other, that are semi-compatible with each other and that are not compatible with each other. As such, as a desired option, feature or step of a selected surgical procedure or technique is entered into a surgical plan, such as based on a patient feature or surgeon preference, the computer database can present other surgical tools and techniques that are compatible with the desired option, feature or step until a full surgical plan can be developed.

A method of planning and preparing for a total knee arthroplasty procedure can comprise: generating three-dimensional models of a tibia and a femur of a patient; sizing the tibia and the femur to within a range based on the three-dimensional models; selecting a resection tool for each of the tibia and femur based on the three-dimensional models; and packaging the resection tools.

A method of planning and preparing for a surgical procedure can comprise: generating a three-dimensional bone model for one or more bones; sizing the one or more bones based on the three-dimensional model; recording a surgical plan based on the three-dimensional bone model; selecting a first surgical tool for the one or more bone based on the surgical plan; and evaluating selection of a second surgical tool based on a performance parameter of the first surgical tool.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a flowchart illustrating a method of determining and providing a plan for a procedure.

FIG. 2 is a display and system for providing input regarding image data.

FIG. 3 is a flowchart illustrating a method of performing a procedure planned in FIG. 1.

FIG. 4A is a schematic view of a three-dimensionally modeled proximal tibia and distal femur.

FIG. 4B is a schematic illustration of a tibia and a femur having an intramedullary rod inserted therein.

FIG. 5A is a perspective view of a patient-specific distal femoral resection guide.

FIG. 5B. is a perspective view of a sensor-assisted distal femoral resection guide.

FIG. 6A is a perspective view of a patient-specific proximal tibia resection guide.

FIG. 6B is a perspective view of a sensor-assisted proximal tibia resection guide.

FIGS. 7 through 17 are various views of an adjustable femoral contour block.

FIG. 18 is a perspective view of a tibial distractor having springs and sensors for performing an alignment check.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

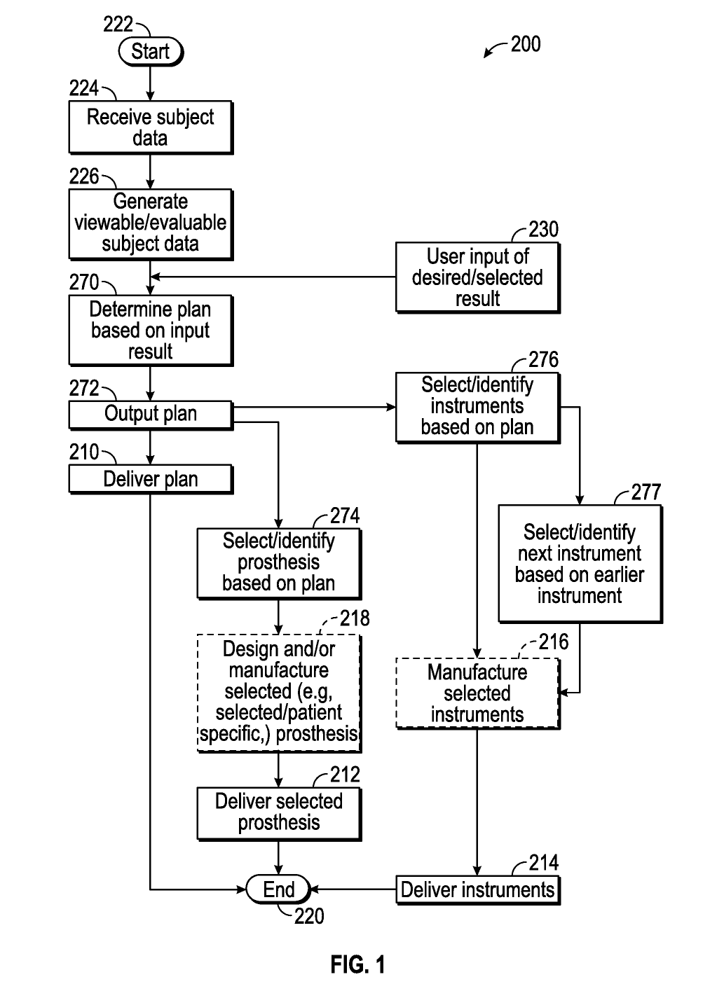

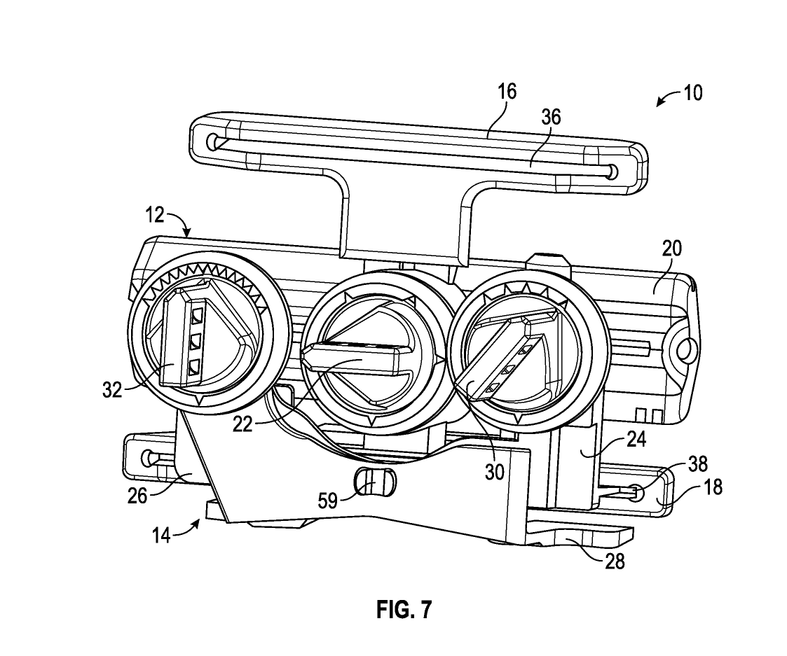

FIG. 1 is a flowchart illustrating a method of determining and providing a plan for a procedure. In general, a surgical plan, as provided herein, can be based on patient data (like-patient (anthropomorphic data), MRI, CT or X-ray) input to create a three-dimensional (3D) model or other information. This can allow sizing (whether general or exact) of the components needed for performing the procedure for a particular patient. In surgery, the distal femoral resection can be performed and positioned, and can be done with a variety of different instruments based on what the surgeon learns in generating the model. An adjustable contour block, illustrated in FIGS. 7-17, can be used after the distal femoral resection to size the implant. A range for the size of the adjustable contour block can be determined from the plan based on the 3D model or the like-patient data, while the actual location and size of the distal femoral resection can be done in surgery. In surgery, the distal femoral resection can be performed and positioned, and can be done with a variety of different instruments based on what the surgeon learns in generating the model and in performing the distal femoral resection. The tibial size or range can be determined by the plan.

With reference to FIG. 1, a flowchart 200 illustrates a process for planning a procedure. It is understood that the planned procedure can be for any appropriate or selected portion of the anatomy. For example, a total hip arthroplasty (THA) can be performed on a subject. THA may include resection of a proximal femoral portion and an acetabulum followed by implantation of prosthetic members for a proximal femur and an acetabulum. Furthermore, total knee replacement, partial knee replacement, shoulder replacement and elbow replacement can also be further examples of prosthetic systems that are implanted into a patient. It is understood that total arthroplasty (generally understood to include replacement of two opposing portions of an articulating surface to completely replace the natural joint) and partial procedures (replacing less than a total articulating portion between two boney sections) can be performed. In various embodiments, a partial knee arthroplasty can include resection or replacement of a medial or lateral condyle of the femur either alone or in combination with a respective medial or lateral portion of the tibia. It is also understood that a partial hip arthroplasty can include resection or replacement of only one portion of a hip joint, such as resection or replacement of a femoral head and/or resection or replacement of an acetabulum or only selected portions of these portions of an anatomy. Similarly, complete or total replacement of any selected portion may be planned and/or performed, including of non-human or living subjects. The following discussion relates generally to a knee arthroplasty, which may include a complete knee arthroplasty. In a complete knee arthroplasty, a distal femur and a proximal tibia can be resected and replaced with prosthetic components. The prosthetic components can interact at the joint to reflect and/or mimic an optimal or selected anatomical interaction. This may include interaction with a pelvis at the hip joint as well.

Initially, the process illustrated in the flowchart 200 can begin at start block 222. It is understood that the method illustrated in the flowchart 200 can be incorporated into instructions that are executed by a processor system. The processor system can include a general purpose processor that is executing instructions that can be stored on a medium, such as a hard disk drive, network memory access, or other memory system. Further, the processor may be a specific processor, such as an application specific integrated circuit (ASIC). According to various embodiments, however, the method illustrated in the flowchart 200 can assist in providing an output for achieving a plan determined by input from a user, such as a surgeon, or based upon instructions within the system. The output may include, as discussed further herein, instrument portions, a list of components for a prosthesis system, and/or instructions to operate or incorporate the instrument or prosthetic portions into a procedure to achieve a planned result. Instructions may include settings for selected instruments, such as guides or sizers. In various examples, the instructions can be included in BiometOS software configured to operate on a handheld or portable electronic device.

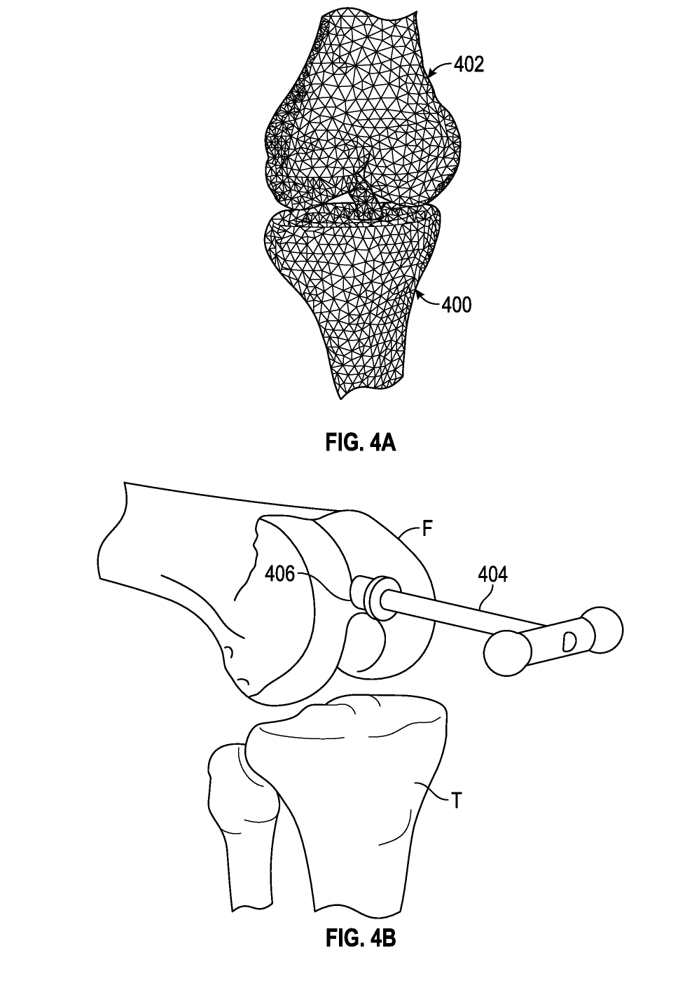

After the method is started in block 222, subject data can be received in block 224. Subject data can be any appropriate subject data such as three dimensional image data or two dimensional image data, or combinations thereof. For example, magnetic resonance images (MRI) that are used to generate three dimensional images of a subject may be incorporated or accessed. Further, or in addition to MRI data, computed tomography (CT) image data and X-ray can be acquired. The CT image data may also be three dimensional image data that is analyzed or used in selecting a plan. Two dimensional image data can also be used to assist in creating a plan, such as by using two substantially orthogonal images to reconstruct a three-dimensional model of a portion of a subject. According to various embodiments, two dimensional image data can be used to generate a three dimensional reconstruction based upon the two dimensional image data. A 3D reconstruction can be created utilizing various software programs such as MIMICS® computer software sold by Materialise N.V. a company in BELGIUM or Amira™ computer software sold by FEI. The software programs are instructions of an algorithm that are executed by a selected processing or processor system. FIG. 4A shows an exemplary 3D reconstruction of femur F and tibia T of FIG. 4B as modeled distal femur 402 and modeled proximal tibia 400.

Further, a two dimensional reconstruction may also be created. The three dimensional reconstruction may be a formed model of the subject based upon the actual image data acquired of the subject. Thus, the model may be a generated, such as a computer generated, image for display with a display device. Two dimensional image data can include two dimensional X-rays that are acquired with selected imaging systems, such as fluoroscopy systems, C-arm imaging systems, and the like.

Additional data regarding the subject can be acquired including anatomical or physical measurements, prior procedures, and the like. The additional patient data may assist in planning a procedure, such as correcting for previous injuries and/or disease. Accordingly, it is understood that data acquired of a subject need not be limited to image data. The additional patient data can be used to check or even select components, such as determining the size of a patient. The additional patient data can include anthropologic data. Anthropometric data are publicly available from many sources and can include, among other things, lengths for body segments, density, mass and inertial properties, and centers of mass and axes of rotation. See, for example, David Winter, Biomechanics and Motor Control of Human Movement, 4th Edition, Chapter 4, Anthropometry, 2009, John Wiley & Sons, Inc. FIG. 4.1 of Winter's book provides, for example, various body segment lengths expressed as a fractions of body height. The Department of Defense maintains a collection of anthropometry resources. See for example “The Body Size of Soldiers: U. S. Army Anthropometry—1966” by Robert M. White and Edmund Churchill, December 1971 at http://www.dtic.mil/get-tr-doc/pdf?AD=AD0743465. In examples, the anthropometric data alone can be used to derive a surgical plan or a portion of a surgical plan, such as by determining a range of sizes, determining instruments, considering ways to review soft tissue, etc.

Regardless of the data collected, following the receiving or the acquisition of data of a subject, the data may be generated for a viewing or evaluation of the subject data in block 226. The generation of the data can include the rendering of the three dimensional reconstruction based upon the two dimensional images. Further, the generation of the viewable data can include display of the three dimensional image data generated with appropriate imaging techniques, such as the MRI. According to various embodiments, however, the data of the subject can be displayed or generated for viewing or evaluation by a user. A user, such as a surgeon, can view the data for determining an appropriate plan. Further, the user may view the data and augment or confirm a plan generated based upon the image data acquired and the data of the subject acquired and evaluated by an appropriate system. The method 200 may be executed by a selected circuit or system, as discussed herein, as a stand-alone feature or it may be incorporated into various planning systems such as the Signature™ Personalized Patient Care System sold by Biomet, Inc. having a place of business in Warsaw, Ind., or the aforementioned BiometOS system.

According to various embodiments, a user can provide input regarding various geometries and identified anatomical portions and a desired or proposed result(s) in block 230. The input in block 230 can be input into the method 200 such as through an input provided with a processing system. The input may be made via a touchscreen, keyboard, or mouse, etc. for inputting results or proposed results into a system to be achieved with the plan via the method 200.

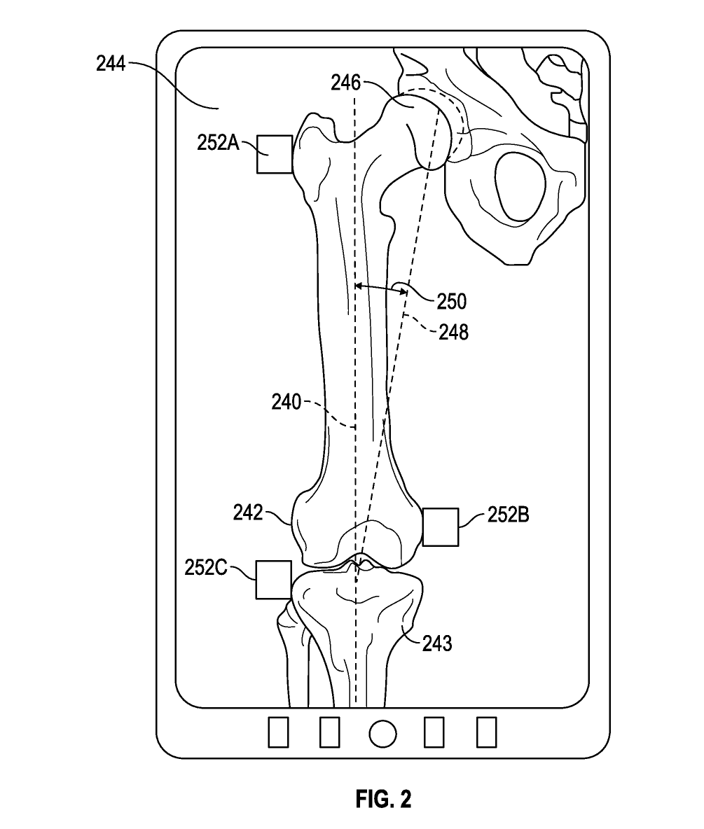

For example, with reference to FIG. 2, a user may identify an axis 240 of a femur 242, such as by drawing a line on a touchscreen display 244 with a finger or non-biological implement. FIG. 2 also shows tibia 243. A mechanical axis 248 extending from a femoral head 246 may also be determined. The mechanical axis 248 can be illustrated and an angle 250 between the mechanical axis 248 and the femoral axis 240 can be calculated or determined. The user, such as the surgeon, can identify or determine the angle 250 as a final angle that can be incorporated into a plan. The user may also augment or change the angle to the final desired or selected angle. The angle 250 may also be referred to as a valgus angle, which is an angle between the mechanical axis 48 and the femoral axis 240. The femoral axis 240 and the angle 250 can also be determined using sensors 252A-252C. Sensors 252A-252C can comprise any suitable sensor, such as position sensors, gyroscopic sensors or radiopaque markers.

It is further understood that analysis of the image data, such as viewed on the display 244, can be used to assist in determining an appropriate size for an acetabular prosthesis, a femoral head prosthesis, a femoral stem prosthesis, and other appropriate portions. It is understood that the selected sizes can be given a certain amount of variability or range such that more than one specific prosthesis component can be provided for a procedure. Thus, two or more sizes may be selected based upon viewing the image on the display 244.

In other examples, the distance between the anterior superior iliac spine (ASIS) of a patient can be used to determine the distance between head centers, as has been established from published information. For example, techniques taught in U.S. Pat. No. 5,885,298 to Herrington and U.S. Pat. No. 5,454,406 to Ritter et al., which are hereby incorporated by this reference in their entirety, can be used to determine anatomic and/or kinematic data useful in planning the procedure. Such data can also be used intraoperatively while performing femoral and tibial resections.

The input from block 230 can allow the user to assist in determining various portions based upon the generated subject data from block 226. The input in block 230 may also allow the user to select results of a procedure. Results may include range of motion, valgus angle, etc. The selected results may be the results of the plan to be determined with the method 220, as discussed herein. The user, therefore, may provide to the system and/or method, medically and/or patient desired results of a procedure. It may also allow a user to provide desired results for a selected procedure, such as placement of a component in an complex mechanical system.

A determined plan in block 270, therefore, may be based, at least in part, on the input in block 230. The determined plan in block 270 can include identifying appropriate prosthetic components, sizes of appropriate prosthetic components, specific instrumentation for achieving a selected result, settings for selected instrumentation, and other appropriate outputs.

The plan can include selecting a final orientation of the anatomical portions, such as a placement and orientation of a femur relative to a pelvis, to achieve a selected outcome. For example, a varus or valgus angle can be selected to achieve a selected orientation of an anatomical portion and/or a range of motion of a joint after an implantation. Based upon the image data of a patient, the placement of various prosthetic components can be determined to achieve the selected varus or valgus angle. It is understood, however, that various other procedures can also be performed with a plan, as discussed further herein. For example, achieving a selected angle (which may also include a varus or valgus angle) of a tibia relative to a femur can be planned. Other anatomical orientations can also be planned for a subject.

Accordingly, various embodiments include outputting a plan in block 272, which may include outputting or identifying selected prostheses in block 274 and/or selecting and identifying instruments based on the plan in block 276. As discussed further herein, the output plan in block 272 can include identifying which prostheses, or a selected range of prostheses, is appropriate for achieving the determined plan in block 270. Further, the output plan in block 272 can include selecting instruments and/or selecting or identifying settings for the instruments in block 276.

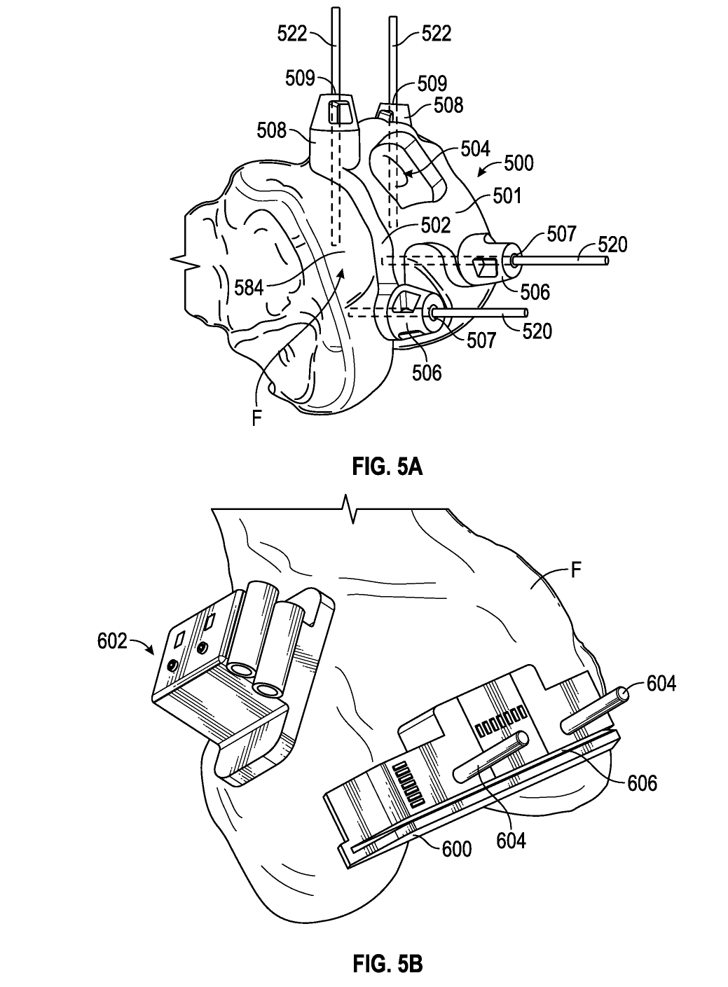

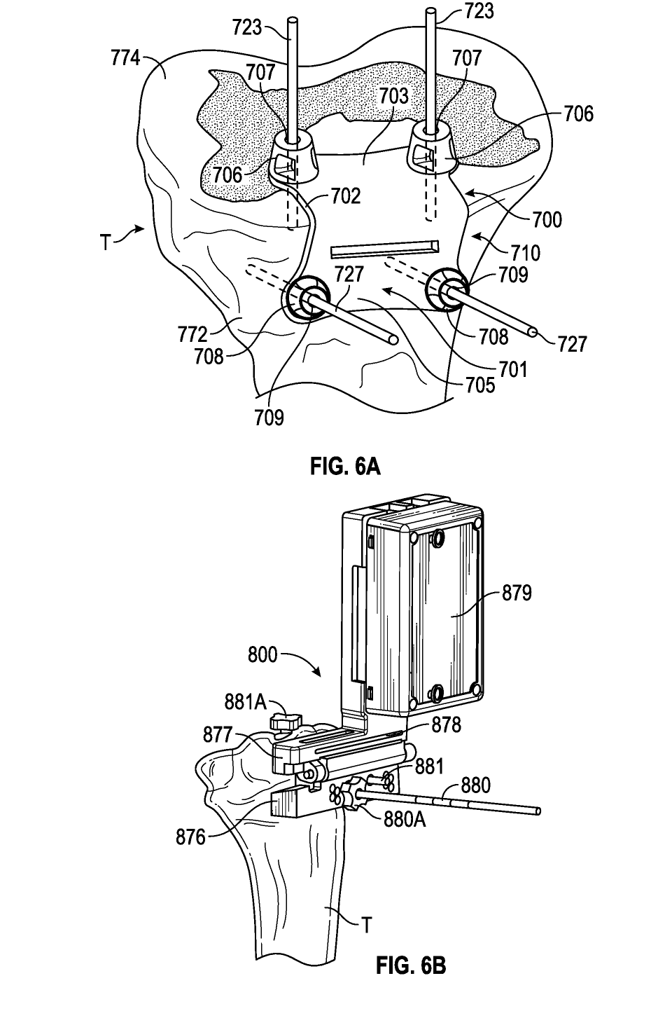

Selecting the instruments in block 276 can also include utilizing a database that can include characteristics of a plurality of different tools and instruments that can perform the same or equivalent tasks, as well as the surgical technique guides that can document procedures for utilizing each tool and instrument. As such, in block 277 the instruments can be selected iteratively wherein a first instrument is selected based on the surgeons preferred starting point in the planning process. For example, a surgeon may decide that the tibia is in a more suitable condition to receive the first resection in a total knee arthroplasty procedure. The surgeon can base that decision on a variety of factors such as bone condition. The surgeon can also consider extension, flexion, anterior cortex, patella, alignment (limb or bone) and tissue balance to position the implants in a knee procedure. The database can then prompt the surgeon with a variety of options available for performing a tibial resection, such as the patient-specific tibial resection guide 700 of FIG. 6A, the sensor-based tibial resection guide 800 of FIG. 6B, conventional tibial resection blocks or others. The surgeon can then decide which instrument is best suited to the particular patient. For example, the surgeon may select the sensor-based tibial resection guide 800 based on input such as patient-specific instruments not being available or the anatomy of the patient not being conducive to patient-specific instrumentation. The database can then suggest suitable instruments for performing the distal femoral resection. It may be that the patient-specific femoral resection guide 500 of FIG. 5A, the sensor-based femoral resection guide 600 of FIG. 5B, conventional cut blocks or others are all suitable, or it may be that for a particular patient, the selection of a sensor-based tibial resection guide renders the use of a patient-specific femoral resection guide unsuitable or cost prohibitive. In any event, the surgeon can input various patient parameters and surgeon preferences at each step and the database can generate a listing of options for other steps in the surgical plan that are viable along with instructions for performing the next step, and a listing of options that are not viable along with an explanation as to why that option is not recommended. The database the may be stored in memory in communication with the processors discussed herein operating with the selected software program such that said database can be viewed on a display screen of a computer or handheld device.

The plan can be output using a process that can include a program that may be executed with a processor (e.g. integrated circuit or application specific circuit) that is executing instructions based on stored software. A plan may then result in instructions that identify portions of an anatomy (or non-anatomical portions for a nonanimal subject) and those portions of the anatomy are identified or localized to a subject. Once the location is identified on the subject according to the plan, an operating instrument, such as a drill or saw, may be positioned and stabilized relative to the identified portion to achieve the planned result. The output plan may be recorded, printed, displayed or published in a variety of manners. For example, the plan may be recorded in memory in communication with the aforementioned processors operating with the selected software program such that said plan can be viewed on a display screen of a computer or handheld device, or the plan may be printed to a physical medium such as paper.

For example, selecting prostheses or identifying prostheses in block 274 based upon the plan output in block 272 can include selecting a specific type of prostheses, such as a knee prosthesis that may include a Vanguard® Knee Prosthesis System or Oxford® Knee Prosthesis system both sold by Biomet, Inc. In selecting the prostheses, specifics may be determined and identified in the plan such as the size, a range of sizes, selected components (e.g., mobile bearing or non-mobile bearing) and other specifics relating to the selected prostheses for achieving the determined plan from block 270.

In various embodiments, the plan can include identifying a location for positioning an intramedullary (IM) rod, such as rod 404 (FIG. 4B) into a femur 242. The position of the IM rod may be determined based upon known or predetermined instrument geometries, such as instruments provided with the Ascent® knee system sold by Biomet, Inc. The determination of the position for the IM rod may be further based upon the determined and/or identified geometry of the anatomy by the user, as illustrated in FIG. 2. Further, the determined location of the IM rod may be based on the selected or identified outcome from block 272.

It is understood that currently available instruments and prosthesis may be included in the previously mentioned accessible database that is used to achieve the plan in block 272. For example, various patient-specific instruments, such as those shown in FIGS. 5A and 6A, or various sensor-assisted instruments, such as those shown in FIGS. 5B and 5B, can be considered in block 272. For example, Signature™ guides commercially available from Biomet, Inc. and iASSIST™ guides commercially available from Zimmer, Inc. may be included in the database. Additionally, other sensor-assisted instruments may be included in the database, such as eLibra® pressure sensing devices commercially available from Zimmer Biomet, as well as conventional instruments. A dynamic knee balancer with pressure sensing is described in U.S. Pat. No. 8,715,290 to Fisher et al., which is hereby incorporated by this reference in its entirety for all purposes.

The known and/or stored geometries, sizes, etc. of both the patient and the various instruments may be used to achieve the result input by the user in block 230. The system that is determining the plan in block 270 may access the database to determine those instruments, prosthesis, etc. to achieve the user input from block 230. The database may be searchable with a relational database with connected relationships. The database may also include surgical techniques for each included instrument.

Thus, based on the determined plan 272 and based upon the selected position of the IM rod 404 within the femur 242, selected instruments can be identified for positioning relative to the IM rod 404 to achieve results based upon the determined plan in block 270. In other words, instruments, such as a cut guide, may be placed on or connected to the IM rod 404 for performing a portion of a procedure. Alternatively, other landmarks, such as biological features, may be used as references for performing the procedure other than an intramedullary rod.

Further, the output plan in block 272 can include selecting instruments based on the plan and/or selecting settings for instruments. For example, with reference to FIGS. 7-17 an integrated instrument 10 having a 4-in-1 cut block and a sizer may be positioned on the femur F. In other examples, a 4-in-1 cut block may be positioned over the IM rod 404, or a 4-in-1 AP chamfer guide sold by Biomet, Inc. with the Ascent™ total knee system, may also be connected with the IM rod 404 in a selected or determined position prior to the instrument 10. Operation of the instrument 10 is described with reference to FIGS. 7-17. Accordingly, the output plan 272 can identify specific and predetermined settings for the instrument 10 to achieve the preselected input results by the user in block 230. It is understood that other appropriate instruments may also be determined, such as a resection block for selecting an amount of a distal resection when positioned on the IM rod 404. Further, or alternatively thereto, patient-specific or custom instruments may also be generated (e.g., by rapid prototyping) based upon the output plan 72, such as the patient-specific distal femoral resection guide 500 of FIG. 5A or the patient-specific proximal tibia resection guide 700 of FIG. 6A. The patient-specific or custom instruments may also be positioned relative to the femur F based upon the IM rod 404 positioned within the femur F, or by the use of pins placed via patient-specific devices. Custom implants or instruments, however, may also be formed to include a surface that will substantially mate with only a determined configuration of a specific patient, such as a bone and/or tissue surface.

The positioned IM rod 404, therefore, may be used to identify a reference point and/or reference location for various instrumentation used for performing a procedure. By having the IM rod 404 positioned within the femur F at the planned location, the instrumentation and/or prostheses can be positioned relative to the femur F to achieve the planned output in block 272. Thus, the input from block 230 that includes the selected result, such as a desired range of motion or valgus angle, may be achieved by performing the procedure based on the determined plan that is output in block 272.

It is understood that other selected instruments may also be provided that allow for selectability. For example, a patient-specific distal femoral sizer may be generated and manufactured based upon the plan determined in block 270. The patient-specific sizer may also be positioned on the IM rod 404, once the IM rod 404 is within the femur F. The patient-specific sizer may be based upon the plan determined in block 270 and may include appropriate orientation and/or contact points to engage the distal femur F prior to any resection. Nevertheless, both a multiple use adjustable system and/or a single-use substantially patient-specific system may be used as the selected instruments based on the plan 276.

Further, the selected prostheses from block 274 may be based upon the plan 270 which is based upon the user input from block 230. The selected prostheses in block 274 can include a size, size range, and type of prosthetic components. For example, for a total knee arthroplasty, a prosthetic system may include a tibial plateau prosthesis, a tibial bearing prosthesis, and a distal femoral prosthesis. Each of these prosthesis components may be provided in several sizes. To achieve an appropriate size for a selected range of patient population (e.g., 99%) several components of various sizes may be required, such as six distinct sizes of each of the three components. Based upon the plan from block 270, however, it can be determined that the specific patient is within a range of one or two sizes of each of the components. Thus, only the selected component sizes may be delivered for a selected procedure. This allows for the procedure to occur with a minimal amount of components for selection by a user and a minimal preparation and related or associated costs for cleaning, manufacturing, or the like of the components.

The output plan in block 272 can also include written or electronically transmitted instructions to a user. The output of the plan in block 72 can identify to the user the selected range of sizes for the components, settings for an adjustable instrument system, and a proposed placement for the IM rod 78. With reference to FIG. 4A, for example, the output of the plan 272 may include a visual illustration of a distal portion of the femur F, as discussed above, with a target or access location 406 indicated thereon. The illustration of the access point 406 may be a target or selected point for insertion of the IM rod 404 into the femur F. The illustration of the distal femur can be based upon the image data generated or accessed by the method 220. The plan output in block 272 may also include a 3D model, 3D image, written instructions, etc.

Thus, a user may be able to identify the target location 406 from the output, illustrated in FIG. 4A, on the specific patient. The target location 406 is determined as part of the plan determined in block 270 to achieve the desired or selected result that is input in block 230. The IM rod 404 may then be inserted into the patient to ensure that the plan is carried out based upon the generated image data and the identified plan.

Further, the output in block 272 may include the determined specific settings, such as locations of the portions of a reusable sizer, for determining an appropriate progression of the plan. Further, the output of the plan 272 may include identification of a portion of a cut block to be used to ensure an appropriate resection of the distal portion of the femur F and other portions of the plan. Thus, the output of the plan for block 272 can allow a user to achieve the results input by the user in block 230 based upon the determined plan block 270.

After the plan has been output in block 272, the prostheses selected in block 274, and the instruments that have been selected in block 276, and any other selection portions, can be delivered. For example, the plan can be delivered in block 210, the selected prostheses can be delivered in block 212, and the instruments can be delivered in block 214. It is understood, according to various embodiments, that the selected instruments can also be manufactured in block 216. As discussed above, the instruments can be based upon the plan output or determined in block 270 for a specific patient (i.e. single patient). Accordingly, the instruments may be manufactured based upon the plan determined in block 270. These instruments may be manufactured in block 216 after the selection of the instruments in block 276. It is understood, however, that the instruments may not be or need not be patient-specific. The output plan in block 272 can include specific settings (e.g. sizes) of generalized instruments that are adjustable for selected procedures. It is further understood, according to various embodiments, that selected prosthesis may also include patient-specific or designed prosthesis that are manufactured or designed in block 218. If the database of prostheses does not include a prosthesis to achieve the input results in block 230 then a patient-specific one may be determined. The patient specific prosthesis may be designed and manufactured in block 218 and then delivered in block 212.

Each of the portions can be delivered according to various commonly known techniques, such as electronic transmission, postal delivery, courier delivery, or other appropriate delivery systems. For example, the plan can be delivered on block 210 via an electronic transmission from a plan provider, such as Biomet, Inc. via known delivery systems including those incorporated with the Signature™ Patient-Specific System or other appropriate delivery systems. The prostheses and instruments can also be delivered in blocks 212, 214 according to appropriate and generally known techniques. The method 200 can then end in block 220.

Ending the method in block 220, however, is understood to possibly precede performing a procedure on a patient based upon the determined plan in block 270. That is, after the plan, prostheses, and instruments are delivered in blocks 210-214 the user may perform a procedure on a selected subject, such as a human patient, with the delivered items. In performing a procedure, the delivered plan from block 210 may be followed and the user can use the delivered instruments from block 214 to implant the delivered prostheses from block 212. As noted above, the delivered instruments from block 214 can be used to achieve the plan output in block 272, which has been determined in block 270. The delivered prostheses in block 212 may allow for the user to select an appropriate prosthetic member from a limited range of sizes, rather than all possible sizes, based upon the determined plan in block 270. Thus, the user can perform a procedure based upon the determined plan 270 to achieve the selected results in block 230.

Generally, therefore, the method illustrated in FIG. 1 may determine a plan to achieve a desired or selected result chosen and input in block 230 by the user. The user may be a surgeon. Based on the input from block 230 along with the generated data/view in block 226, the system may determine the plan in block 270. In determining the plan the system may access the database of known and/or available instruments along with known and/or available prostheses. Each of these known and/or available instruments along with known and/or available prostheses may be analyzed using their known geometries and sizes to determine the plan in block 270 that would achieve or best achieve the input result from block 230. For example, the input desired result may be a selected range of motion (ROM) and valgus angle. The system may then analyze the generated subject data and the database of instruments and prostheses. The system may then determine which instruments, which specific settings for the instruments, and which specific prosthesis(es) may achieve the input result. The system may then determine the plan in block 270 and output the plan in block 272 including the identified and selected instruments, settings, and prostheses. Also, the system may determine designs for instruments and prostheses, if the database does not include appropriate instruments and/or prostheses. All of this may occur prior to the beginning of a procedure, such as before placing a subject in an operating room. Thus, instrument and prosthesis selection may precede initiating a surgical procedure. Further, the user, such as a surgeon, may only be required to input a selected result and the system determines the plan to achieve the result.

In addition to the illustrated method 200, various instruments and devices may be used to assist in a procedure. For example, various guides and templating instruments can be positioned relative to a portion of anatomy, such as the distal femur F, for performing a procedure. According to various embodiments, the instruments can be positioned relative to the distal femur and adjusted according to a predetermined plan. For example, the method 200 can be used to assist in identifying or positioning a template or instrument relative to a distal femur based upon settings provided to a user, similar to those discussed above.

According to various embodiments, the distal portion of the femur F may be prepared or oriented based upon a predetermined plan, such as the plan illustrated in FIG. 1, using an adjustable femur contour block illustrated in FIGS. 7-17. The integrated instrument 10 can be oriented relative to femur F as discussed further herein, including the distal portion of femur F. Generally, the integrated instrument 10 can be adjusted relative to the femur F for orienting various portions to be positioned relative to the femur and/or to sections of the femur F. For example, a rod or drill holes can be formed into the femur for performing various resections of the femur including a distal, proximal, posterior resections, and the like.

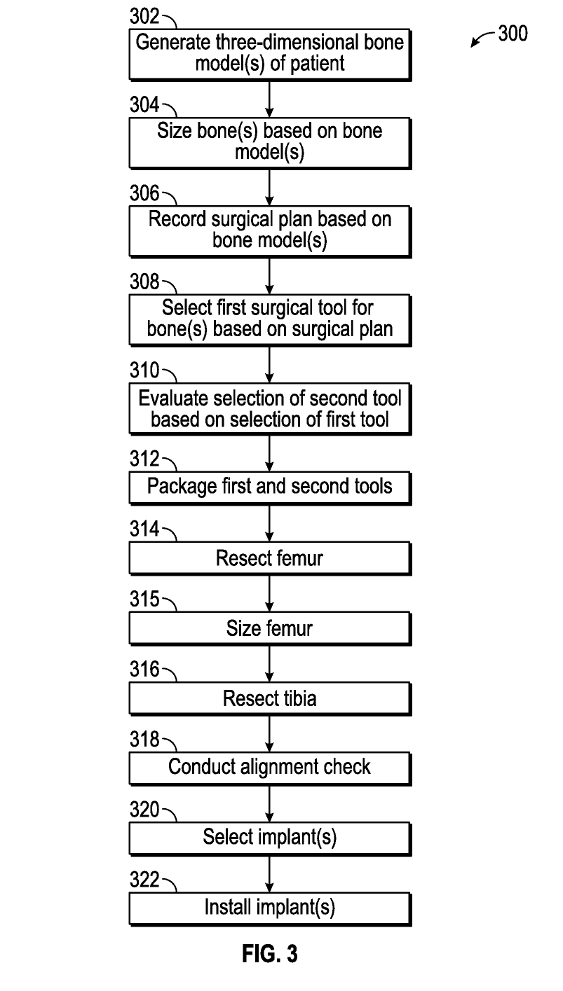

FIG. 3 is a flowchart illustrating a method 300 of performing a procedure, as can at least partially be planned according to the flowchart shown in FIG. 1. The method 300 can begin at block 302 wherein three-dimensional models of bones of the patient can be generated. As discussed above, the surgical plan can be based on patient data including like-patient data, MRI, CT or X-ray input, with the production of one or more 3D models resulting, or simply instruction based on the like-patient data without X-ray input. The 3D models can allow for sizing of the patient, at least to a range, in block 304. Similarly, the like-patient data can be used to sizing of the patient, at least to a range. In other words, the sizing need not be exact, but can be useful in narrowing the sizes to a range. The 3D models can be generated as described with reference to FIG. 4A, for example.

At block 306, portions of the surgical plan based on the sized bones can be recorded, as described herein. Blocks 308 and 310 indicate the iterative process of selecting surgical instruments and tools discussed above with reference to blocks 276 and 277 of FIG. 1. At block 312, some or all of the selected tools and instruments can be packaged in a sealed, sterile container in advance of the surgical procedure. As such, the packaged surgical tools and instruments can be delivered, as is described with reference to block 214 in FIG. 1.

Once the surgical procedure begins, the femur F can be resected in block 314 and the tibia T can be resected in block 316. However, in other examples or procedures, the tibia T can be resected first and the femur F can be resected second.

In block 314, the resection of femur F can be accomplished by the Signature Guide (e.g., resection guide 500 in FIG. 5A), standard instruments or i-Assist (e.g., resection guide 600 in FIG. 5B). The benefit of a sensor-assisted instrument is that it can determine the femoral head center at time of surgery, when it cannot be seen. Additionally, if the distance between the ASIS (Anterior Superior Iliac Spine) of a patient is known, the distance between head centers could be established from published information, such as by using the technique described above with reference to the Ritter et al. patent.

Also, the contra-lateral head center can be obtained thru sensors, such as by using sensors 252A-252C of FIG. 2. Also, these dimensions can be estimated thru patient data like height and weight or other details, such as anthropomorphic data. Knowing the contra-lateral head center distance for the patient and the length of the limb (which can be measured or estimated from the head center to the joint line to the ankle) can give the kinematic (actual) joint line angle from the mechanical axis (half the head center distance divided by the limb length basically gives the tangent of the kinematic angle). Additionally, sensors 252A-252C of FIG. 2 can be used the tibial T and the femur F and the knee joint could be flexed and extended to determine the kinematic axis. Knowing this angle allows for consistent orientation of cuts (angle from the mechanical axis could be replicated on the femur in extension and flexion and tibia) for all joint line orientations and could be patient specific.

At block 315, the femur F can be sized. In one example, the adjustable contour block described with reference to FIGS. 7 through 17, integrated instrument 10, can be used after the distal femoral resection is performed. A range for the size of integrated instrument 10 can be determined from the plan, such as from the 3D models or from like-patient data, with the actual size being determined during surgery. Integrated instrument 10 can be used to specifically size and position the femur F in surgery after the distal resection is performed, as described with reference to FIG. 7 through 17. An additional feature can be to use the joint line orientation determined in extension for flexion (kinematic or perpendicular) to help orient the integrated instrument 10. This can be a manually set calibration or utilize a sensor, potentially paired with the distal or tibial sensors.

A built-in adjustable medial/lateral width gage can be integrated into another example of integrated instrument 10. For example, medial shim 48 and medial foot 52 can be configured to be adjustably positioned relative to lateral shim 50 and lateral foot 54 such that the medial/lateral width can be read from a scale provided on integrated instrument 10. Alternatively, space could be included in integrated instrument 10 to receive a plug-in medial/lateral width checker. The medial/lateral width can be weighted as a concern during sizing of the femur F.

At block 316, the tibia T can be resected during surgery. This resection can be accomplished by the Signature Guide (e.g., resection guide 700 in FIG. 6A), standard instruments or i-Assist (e.g., resection guide 800 in FIG. 6B). The resection can be Kinematic or perpendicular to the mechanical axis in the medial/lateral direction, and it can be perpendicular or include various, posterior slopes in the anterior/posterior direction.

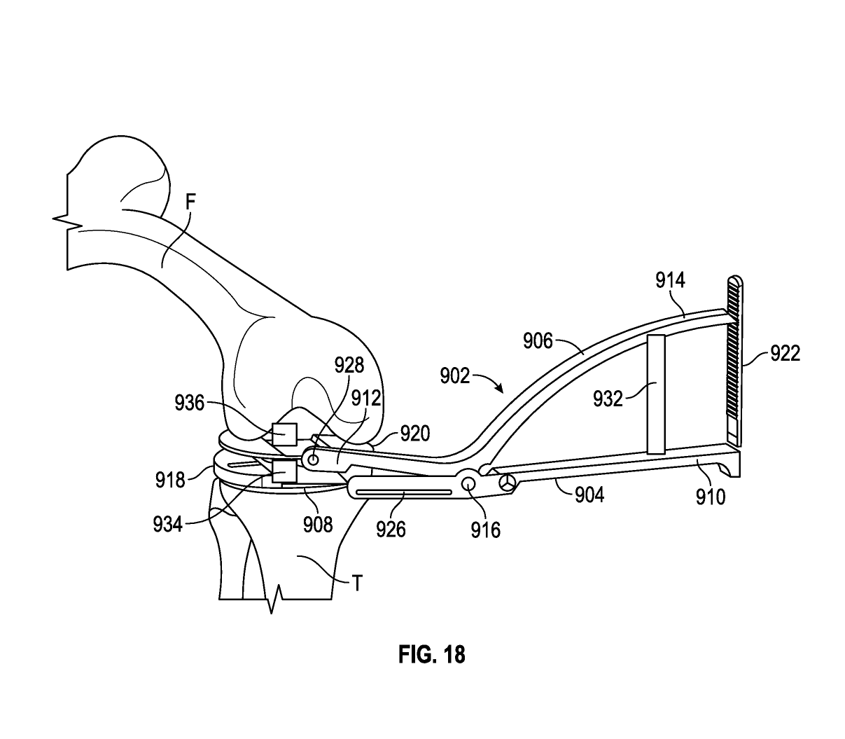

At block 318, the femoral and tibial resections can be positioned relative to each other thru a distractor device, such as the distractor instrument 900 of FIG. 18 or another device. This can be a non-calibrated or calibrated device, and can include commercially available products as described herein such as the eLibra device or the OrthoSensor device. This check can allow soft tissue modification, bone resection alteration or just be a reference, depending on the surgeon and patient specifics.

Also, knowing the mechanical alignment from the plan and surgery details can allow orientations to account for specifics based on the patient data. Utilizing a distractor after one of the bone resections to place another also can allow for relative orientation and distance to be set. Additionally, the calibrated distraction (device or sensors) can clarify the makeup of the medial/lateral load. Rather than a 50%/50% split, the load might be split 60%/40% (or potentially desired to be in a range, like 50/50 to 70/30, based on anthropometric data). It might even mean the distraction can be done with one condyle controlled and the other a follower, allowing the alignment (or some other anatomy reference) to set the tissue balance and cut orientations and positions. This can effectively allow evaluation of alignment, balance and location (potentially in flexion and extension) to decide appropriate resections. Finally, the cuts could be reviewed based on overall alignment, and accepted if the hip center to ankle line is within the medial/lateral condyle contacts of the knee femoral, this being potentially, inherently stable without soft tissue balance.

After a first resection is performed, such as on the femur F, the following method can be used to create an extension or flexion space with unequal medial and lateral gaps or unequal soft tissue loads. The joint space can then be tensed with equal load on the medial and lateral soft tissue. A second bone cut, such as on the tibia T, can be made non-parallel to the first cut resulting in a smaller (or larger) medial joint space relative to the lateral joint space.

The size range of tibia T can be determined in the plan as described above. The plan would reduce the sizes needed to actually do a surgery to a range, at least. Also, the surgical plan can help clarify orientations of resection guides for tibia T.

In blocks 320 and 322, the implants can be selected based on information determined from blocks 314 to 318 and the surgical plan and subsequently implanted or installed in a conventional manner.

FIG. 4A is a schematic view of a three-dimensionally modeled proximal tibia 400 and distal femur 402. FIG. 4B is a schematic illustration of a tibia T and a femur F having an intramedullary rod 404 inserted therein.

In one example, the modeled proximal tibia 400 and distal femur 402 may be three-dimensionally modeled using the systems and methods described in U.S. Pat. No. 8,884,618 to Mahfouz, which is hereby incorporated by this reference in its entirety for all purposes. Tibia 400 can be a three-dimensional model of tibia T, and femur 402 can be a three-dimensional model of femur F. Femur F can be femur 242 and tibia T can be tibia 243 from which the surgical plan was developed with reference to FIG. 2.

Planning is done before actual implant placement is defined on the bone. It includes interactively determining where a final position of an implant is to be relative to patient specific landmarks. Landmarks may be identified during the plan, such as identifying landmarks (hard or soft tissue or axis, e.g. femoral epicondyles) on tibia T, femur F, modeled proximal tibia 400 and/or modeled distal femur 402. The final position may include how an implant will be positioned to achieve selected and planned axes of patient bones, range-of-motion, and other selected results following implantation of a prosthesis. It may also include positioning of any appropriate or selected member relative to a subject or device. The plan may include various results (e.g. varus and valgus angle) based upon resection preparation that leads to prosthesis placement. The plan may be executed by a processor operating with selected software programs, including those discussed above. Further, the plan may be determined by evaluating a X-ray image and constructing a 2D template that may be used to identify landmarks and develop the plan.

During a procedure, such as placement and implantation of a prosthesis it may be desirable and/or necessary to identify on a bone the same datums or anatomic landmarks used in making and determining the plan, as is described with reference to FIG. 2. To ensure that a procedure, such as a resection, matches the plan, the landmarks identified or used by the plan may be identified during a procedure. These may include locating landmarks (hard or soft tissue or axis, e.g. femoral epicondyles). The landmarks may be used for proper resection and/or guide placement. Thus, during a procedure identifying and locating the landmarks may occur and may be referred to as localizing (registration) with the patient bone or other appropriate anatomy.

In one example, such as distal femoral prosthetic placement, the IM rod 404 may be one datum or landmark on the femur that both indicates an anatomic axis (IM canal) and generally provides a stable platform for an instrument in two axes. The IM rod 404 can stabilize things in both a X and an Y axes, but not a Z (rotational) axis. Other landmarks may include the distal femoral posterior condyles which combined with the IM rod 404 gives a rotational datum. The Z datum can be touching the most distal femoral condyle with a guide portion. Another datum may be an anterior cortex of the femur. For a tibia a datum may include an anterior face of the tibia or either side of the tibial tubercle. The width of the femur (or tibia) could also be used. It could be useful to check the articular cartilage low (thinnest) point, either tibial or distal femur. Once these datum are identified, the patient specific parameters from the plan can be applied relative to them. These datum allow the instruments to be positioned relative to the bone through the same datums as the plan, and then stabilized such that they don't move. An instrument may also include patient-specific portions or members, as discussed herein (including a stylus, a patient-specific key or member, a patient specific setting, or a programmable or programmed portion that may alter an instrument to a specific setting) can be inserted into, attached to, or adjusted relative to other instrument portions to set guides in a position to orient the implants identical to the plan. Patient-specific portions or settings can alternatively be applied prior to the other instrument portions being positioned and stabilized.

Once the landmarks are located, an instrument and/or guide, as discussed further herein, may be oriented or placed on and/or aligned with the landmarks. Positioning of an instrument may, therefore, occur based upon the plan. The landmarks identified, as noted above, may be used to orient an instrument, such as a drill or a cut guide, so that it facilitates shaping the bone to position the prosthesis consistent with the plan. Thus, the guides and instruments may be oriented based on the plan to achieve the selected results.

FIG. 5A is a perspective view of a patient-specific distal femoral resection guide 500. An exemplary patient-specific distal femoral resection guide is described in U.S. Pat. No. 8,591,516 to Metzger et al., which is hereby incorporated by this reference in its entirety for all purposes.

Referring to FIG. 5A, an exemplary patient-specific femoral alignment guide 500 according to the present teachings is shown mounted on the corresponding distal femur F of the patient. The femoral alignment guide 500 can have a light-weight body 501 with a patient-specific engagement surface 502 that is complementary and made to closely conform and mate with a portion of the anterior-distal surface 584 of the patient's femur F based on the pre-operative plan, as described above. For example, the engagement surface 502 can be a mirror image of the surface 584. The femoral alignment guide 500 can include a window/opening 504 and first and second distal guiding formations 506 defining guiding bores 507 for guiding corresponding distal alignment pins 520. The femoral alignment guide 500 can also include first and second anterior guiding formations 508 defining guiding bores 509 for guiding corresponding anterior alignment pins 522.

Pins 520 and 522 can remain in femur F so that other patient specific guides can be mounted to femur F in a precise matter using pins 520 and 522, with or without femoral alignment guide 500 being mounted to femur F. For example, a distal cutting block (not shown) can be mounted over the anterior alignment pins 522, which can pass through corresponding openings of the distal cutting block, while the femoral alignment guide 500 is still nested on the distal femur F. The alignment guide 500 can be disposable and made of polymeric material that can also be sawn or cut off. The distal cutting block can include a cutting slot or other cutting guiding formations. A cutting blade can be guided through the cutting slot and can make the distal resection of the femur F sawing through the alignment guide 500 and the alignment pins 520 to create a resected distal surface.

FIG. 5B is a perspective view of a sensor-assisted distal femoral resection guide 600. An exemplary sensor-assisted distal femoral resection guide is described in U.S. Pat. No. 8,265,790 to Amiot et al., which is hereby incorporated by this reference for all purposes.

Resection guide 600 can include tracker member 602, which can be separately secured to femur F via any suitable means, such as pins or fasteners. Tracker member 602 can include various position sensors, such as micro-electromechanical sensors (MEMS), gyroscopes, accelerometers or the like that detect orientation changes in the positioning of femur F, tracker member 602 and/or resection guide 600, and that provide output to the surgeon in aligning resection guide 600 with femur F. Resection guide 600 can be secured to femur F via pins 604 that can be placed using any data from tracker member 602. Once suitable parameters are attained (e.g., varus-valgus, flexion-extension, etc.), the resection guide 600 can be anchored to the femur F, for instance using the pins 604. Subsequently, slot 606 can be used to in conjunction with a resection tool, such as a saw to perform a distal resection of femur F.

FIG. 6A is a perspective view of a patient-specific proximal tibia resection guide 700. An exemplary patient-specific proximal tibia resection guide is described in U.S. Pat. No. 8,591,516 to Metzger et al., which is hereby incorporated by this reference for all purposes.

Referring to FIG. 6A, a representative tibial alignment/resection guide 700 is illustrated according to the present teachings. The tibial alignment guide 700 can include a body 701 having a proximal portion 703, an anterior portion 705 and a patient-specific bone engagement surface 702 complementary and made to closely conform and mate with a portion of the anterior surface 772 and proximal surface 774 of the tibia T of the patient in only one position based on the pre-operative plan. For example, the engagement surface 702 can be a mirror image of the surfaces 772 and 774. The tibial alignment guide 700 can include first and second proximal guiding formations 706 defining guiding bores 707 for corresponding proximal alignment pins or other fasteners 723. The tibial alignment/resection guide 700 can also include first and second anterior guiding formations 708 defining guiding bores 709 for corresponding anterior alignment pins or other fasteners 727. As discussed above in connection with alignment guides in general and the femoral alignment guide 500 in particular, the tibial alignment guide 700 can be used to drill reference holes for the corresponding proximal and anterior alignment pins 723, 727, which can then be re-inserted as needed for each resection and corresponding resection block after the tibial alignment/resection guide 700 is removed. In the embodiment illustrated in FIG. 6A, the tibial alignment/resection guide 700 can include a resection guiding slot 710 for guiding a tibial resection according to the pre-operative plan for the patient. The tibial alignment/resection guide 700 can be optionally used as a resection guide for resecting the tibia T through the guiding slot 710 with a blade or other resection tool while the tibial alignment/resection guide 700 is mounted on the tibia T.

FIG. 6B is a perspective view of a sensor-assisted proximal tibia resection guide 800. An exemplary sensor-assisted proximal tibia resection guide is described in U.S. Pat. No. 8,265,790 to Amiot et al., which is hereby incorporated by this reference for all purposes.

The resection guide 800 can have a base 876 that can be fixedly secured to the tibia T. A cutting guide 877 can be pivotally mounted to the base 876 by a pivot joint. The cutting guide 877 can have a slot 878 into which a blade is inserted to perform cuts on the tibia T. A MEMS unit 879 can be integral with the cutting guide 877 so as to track the orientation of the cutting planes, and provides three-degree-of-freedom tracking to provide tracking data related to the orientation of the cutting guide 977. The resection guide 800 can be secured to the bone by a first threaded rod 880. Once a desired varus-valgus orientation is reached using knob 880A, rod 881 is used so as to secure the base 876 to the bone in the varus-valgus orientation. The flexion-extension orientation is then adjusted using knob 881A so as to reach a desired orientation of the cutting guide 877 in view of creating the cutting planes on the tibia T. It is pointed out that the virtual cut planes may be tracked as a function of the geometry of the slot 878 in the resection guide 800. More specifically, the MEMS unit 875 may be provide with the data representing the cut planes, such that secondary cut planes can be tracked to simulate the positioning of an implant on the bone.

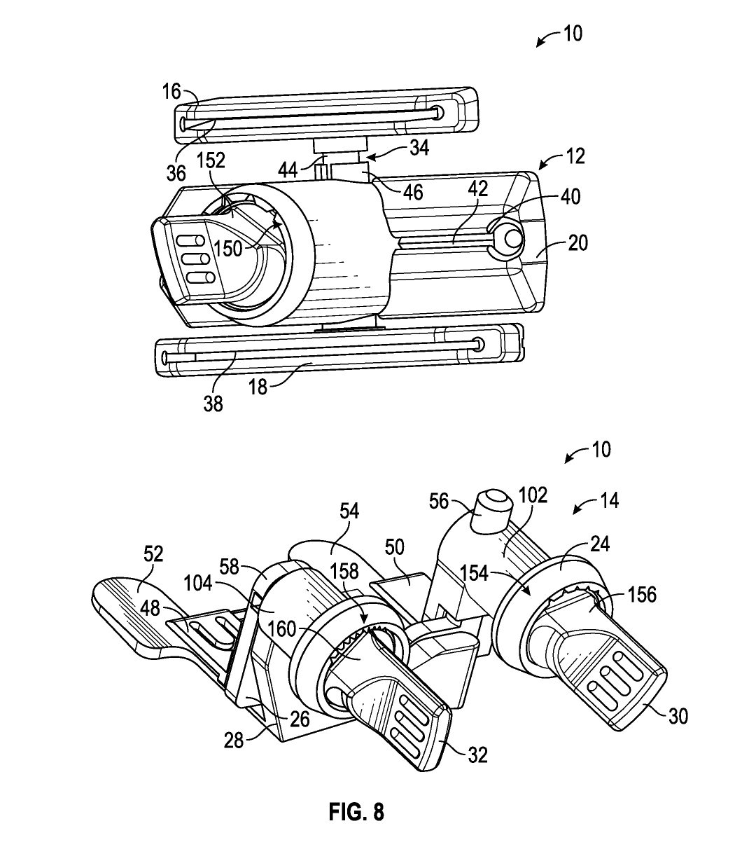

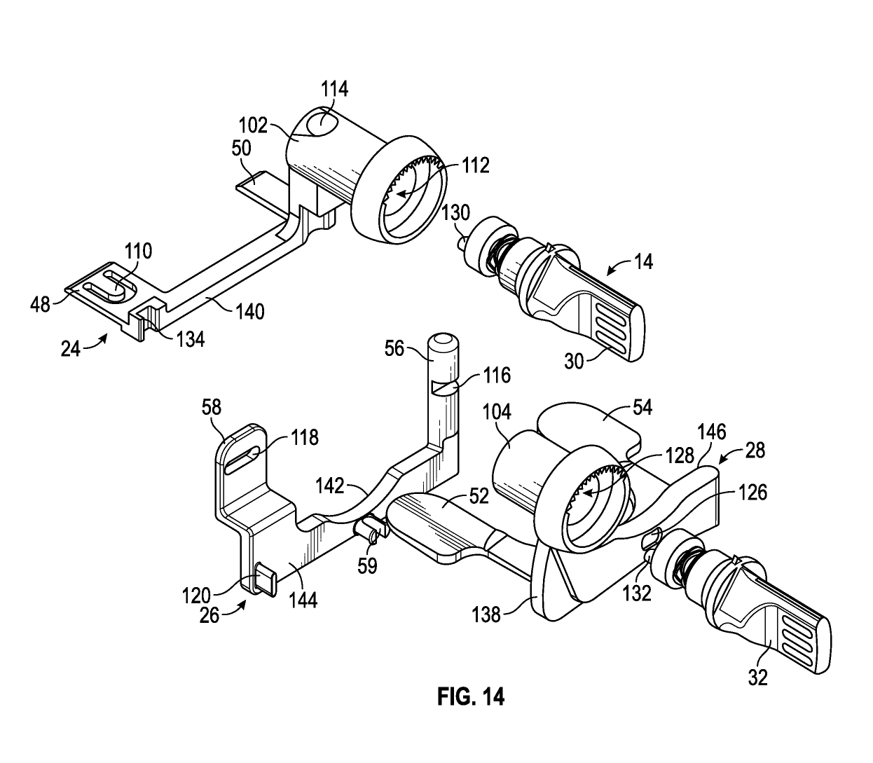

FIG. 7 is a perspective view of integrated instrument 10 for arthroplasty planning having adjustable 4-in-1 cut block 12 and a portion of adjustable sizer 14. FIG. 8 is an exploded view of integrated instrument 10 of FIG. 7 showing adjustable 4-in-1 cut block 12 and adjustable sizer 14. FIGS. 7 and 8 are discussed concurrently. Adjustable sizer 14 is typically used with a stylus that provides a contact for a posterior portion of the femur, as discussed below with reference to FIG. 14.

Adjustable 4-in1 cut block 12 can include anterior cut guide 16, posterior cut guide 18, chamfer block 20 and first adjuster knob 22. Adjustable sizer 14 can include shim body 24, adjuster body 26, foot body 28, second adjuster knob 30 and third adjuster knob 32.

Anterior cut guide 16 and posterior cut guide 18 can be configured to adjust their relative positions about adjustable post 34 using first adjuster knob 22. Anterior cut guide 16 and posterior cut guide 18 can include anterior cut slot 36 and posterior cut slot 38, respectively. Chamfer block 20 can be mounted to adjustable post 34 and includes anterior chamfer slot 40 and posterior chamfer slot 42. Adjustable post 34 comprises anterior post 44 and posterior post 46.

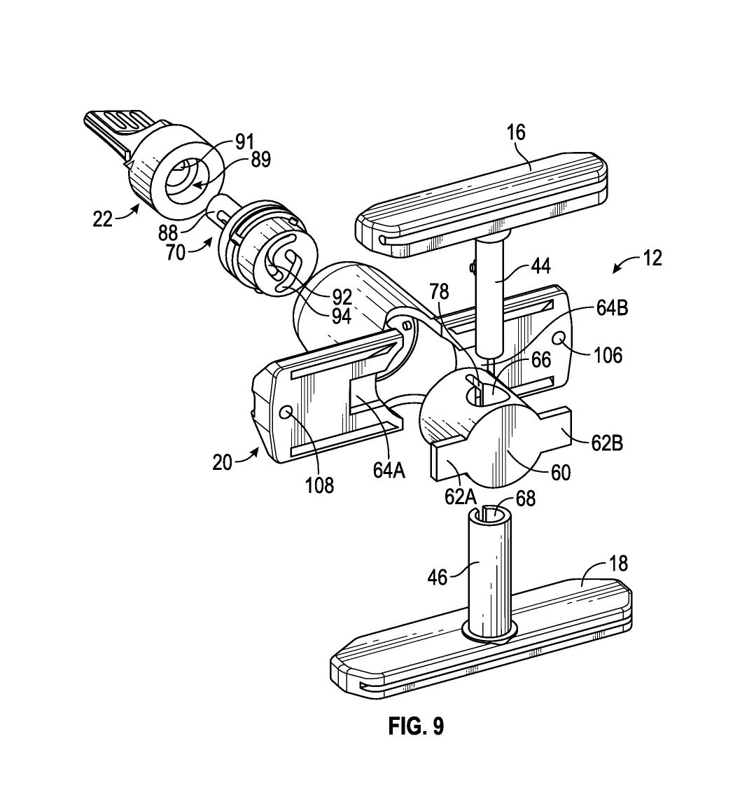

Anterior and posterior cut slots 36 and 38 can be configured to align a cutting device, e.g. a saw blade, with anterior and posterior portions of femoral condyles to facilitate making anterior and posterior resections of the bone. Anterior and posterior chamfer slots 40 and 42 can be configured to align a cutting device, e.g. a saw blade, with anterior and posterior portions of femoral condyles to facilitate making chamfer resections between the anterior and posterior resections and a previously made distal femoral resection. As discussed in greater detail below with reference to FIGS. 9-13, first adjuster knob 22 can engage anterior post 44 and posterior post 46 of adjustable post 34 to control a distance between anterior cut guide 16 and posterior cut guide 18, which can be set relative to a fixed location located therebetween.

Second adjuster knob 30 can be configured to adjust the distance between shim body 24 and foot body 28 using adjuster body 26. Third adjuster knob 32 can be configured to adjust the angular relationship between shim body 24 and foot body 28 using adjuster body 26.

Shim body 24 can include medial shim 48 and lateral shim 50, which can be configured to be inserted into posterior cut slot 38. Foot body 28 can include medial foot 52 and lateral foot 55, which can be configured to engage a proximal end of a tibia or a spacer disposed thereon, as well as the posterior femur for relative position of the cuts to femoral anatomy. As discussed in greater detail below with reference to FIGS. 14-17, second adjuster knob 30 engages a notch in slide pin 56 to adjust the distance between shims 48 and 50 and feet 52 and 54, and third adjuster knob 32 can engage a slot in tab 58 to adjust the angular relationship between shims 48 and 50 and feet 52 and 54, such as at pivot point 59.

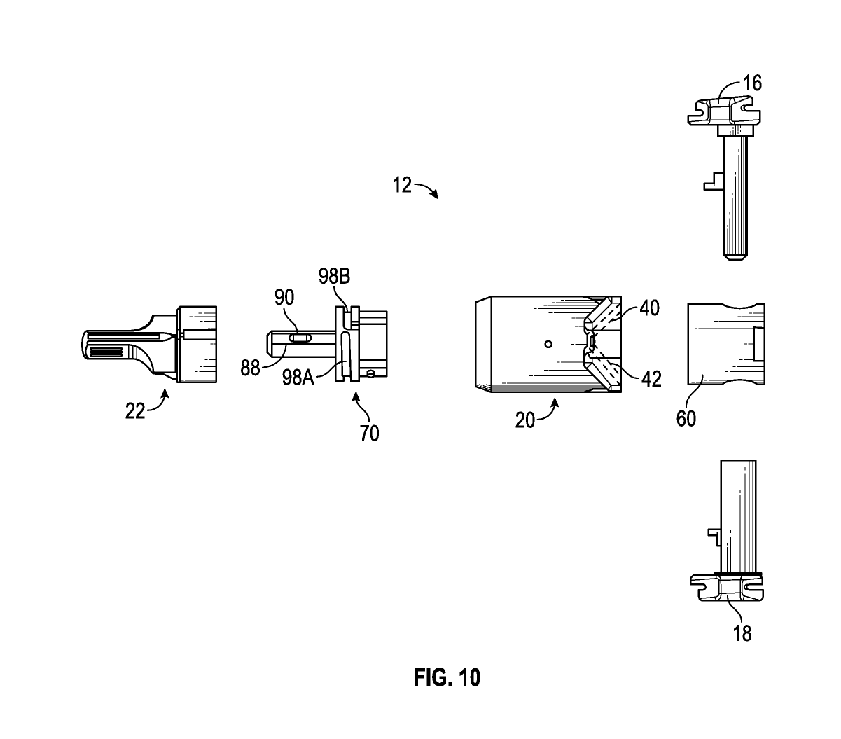

FIG. 9 is an exploded perspective view of the adjustable 4-in-1 cut block 12 of FIG. 8 showing anterior cut guide 16, chamfer block 20 and posterior cut guide 18 connected to main body 60. FIG. 4 is an exploded side view of adjustable 4-in-1 cut block 12 of FIG. 8 showing first adjuster knob 22 aligned with drive pin 70 and main body 60. FIGS. 9 and 10 are discussed concurrently.

Anterior cut guide 16, posterior cut guide 18 and chamfer block 20 can be mounted to main body 60. Main body 60 can act as a reference point from which movement of anterior cut guide 16, posterior cut guide 18 and chamfer block 20 is related. Main body 60 can include wings 62A and 62B that are fitted into sockets 64A and 64B in chamfer block 20 to prevent relative rotation therebetween. The specific size of wings 62A and 62B and sockets 64A and 64B, including width, depth and thickness, can vary in different examples of the device. Main body 60 can also include bore 66 into which posterior post 46 is inserted. Posterior post 46 can include bore 68 into which anterior post 44 is inserted to form adjustable post 34 (FIG. 2). Anterior chamfer slot 40 and posterior chamfer slot 42 can be seen in phantom extending through chamfer block 20 in FIG. 10.

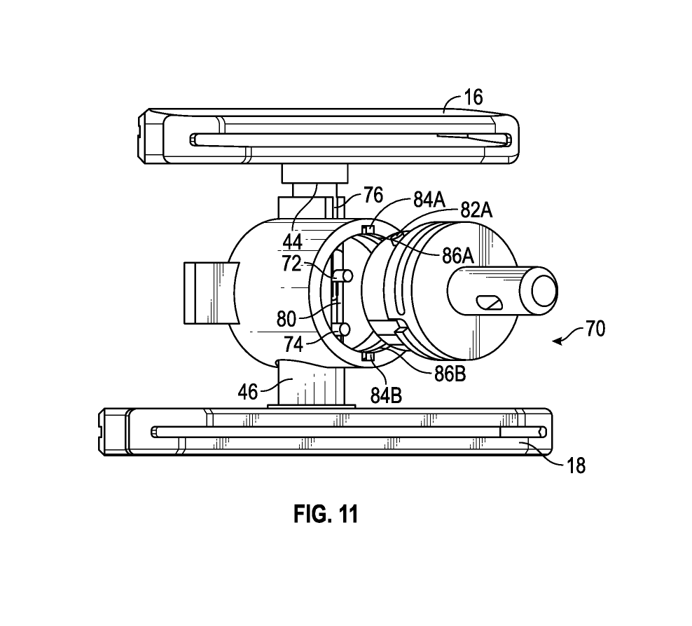

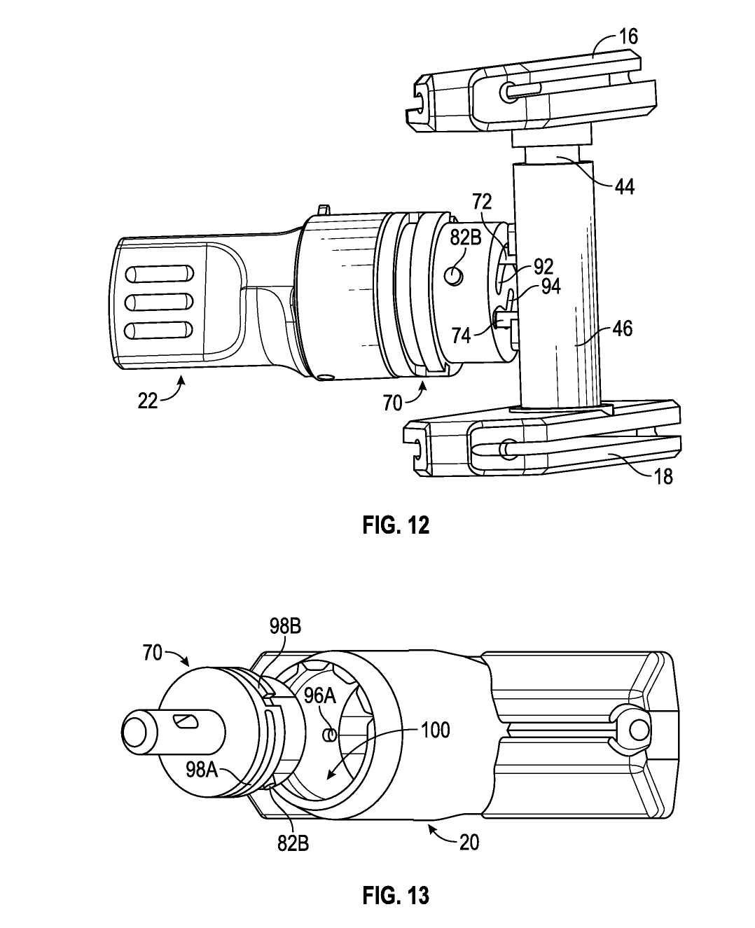

First adjuster knob 22 can be connected to drive pin 70, which passes through chamfer block 20 (as can be seen in FIG. 13) to be inserted into main body 60 (as can be seen in FIG. 11) to couple with anterior post 44 and posterior post 46 (as can be seen in FIG. 12).

FIG. 11 is a partial assembled view of adjustable 4-in-1 cut block 12 of FIG. 8 with chamfer block 20 and first adjuster knob 22 removed from drive pin 70 to show posts 44 and 46 of anterior and posterior cut guides 16 and 18, respectively, disposed within main body 60 to form adjustable post 34. Anterior post 44 can include anterior finger 72, and posterior post 46 can include posterior finger 74.

Posterior post 46 can include cutout 76 to accommodate finger 72. Likewise, main body 60 can include cutout 78 (FIG. 9) to accommodate fingers 72 and finger 74. The cutouts of main body 60 can connect to slot 80 in which fingers 72 and 74 translate when actuated by drive pin 70.

Drive pin 70 can be inserted into main body 60 by aligning protrusion 82A with notch 84A that connects to channel 86A in main body 60. Main body 60 can also include notch 84B and channel 86B for mating with protrusion 82B (FIG. 12). Drive pin 70 can be coupled to first adjuster knob 22 (FIG. 10) via insertion of shaft 88 into complimentary bore 89 (FIG. 9) within knob 22. A pin or other fastener 91 (FIG. 9) can be inserted through knob 22 and hole 90 in shaft 88 to prevent relative rotational movement between drive pin 70 and knob 22. A spring, can be positioned around shaft 88 to bias knob 22 away from drive pin 70. Thus, rotation of knob 22 by an operator of instrument 10 can cause drive pin 70 to rotate, which in turn can cause posts 44 and 46 to be drawn toward or pushed away from each other while remaining proportionally spaced from main body 60.

FIG. 12 is a partial assembled view of adjustable 4-in-1 cut block 12 of FIG. 8 with chamfer block 20 removed to show fingers 72, 74 of posts 44, 46 disposed within slots 92, 94, respectively, of drive pin 70. FIG. 12 also shows protrusion 82B of drive pin 70.

Drive pin 70 can operate as a cam wheel to produce movement of posts 44 and 46. As can be seen in FIG. 9, slots 92 and 94 are irregularly shaped such that rotation about a central axis extending through shaft 88 of drive pin 70 can cause linear movement of fingers 72 and 74. Slots 92 and 94 can be differently shaped to produce different rates of travel of posts 44 and 46.

As drive pin 70 rotates, protrusions 82A and 82B can retain drive pin 70 within channels 86A and 86B of main body 60. Drive pin 70 can be retained within chamfer block 20 using a similar protrusion and channel connection.

FIG. 13 is a perspective view of drive pin 70 exploded from chamfer block 20 to show alignment tab 96A of chamfer block 20 and alignment slots 98A and 98B of drive pin 70. Drive pin 70 can be inserted into chamber 100 of chamfer block 20 so that alignment tab 96A engages alignment slot 98A. Likewise, alignment tab 96B (not shown) opposite alignment tab 96A within chamber 100 can engage alignment slot 98B. Slots 98A and 98B can allow rotation of pin 70 within chamber 100, but can also control axial movement when fully engaged. As can be seen in FIG. 10, slots 98A and 98B vary their distance from the ends of pin 70 to allow chamfer block 20 to move relative to main body 60, while protrusions 82 keep pin 70 fixed relative to main body 60. Furthermore, with pin 70 fully seated in chamfer block 20, knob 22 can be coupled to protrusions 82A and 82B to further prevent axial displacement of drive pin 70 from chamber 100.

With reference to FIG. 8, as will be discussed in greater detail later, rotation of knob 22 can extend and retract anterior cut guide 16 and posterior cut guide 18 from chamfer block 20. In one example, right-hand rotation of knob 22 can push anterior cut guide 16 and posterior cut guide 18 away from chamfer block 20 to allow for wider anterior and posterior resectioning of larger femurs, while left-hand rotation of knob 22 can pull anterior cut guide 16 and posterior cut guide 18 closer to chamfer block 20 to allow for narrower anterior and posterior resectioning of smaller femurs. Once anterior cut guide 16 and posterior cut guide 18 are positioned in a desired location, anterior chamfer slot 40 and posterior chamfer slot 42 are additionally properly positioned, e.g. between anterior cut guide 16 and posterior cut guide 18, to allow for chamfer resectioning of the femur.

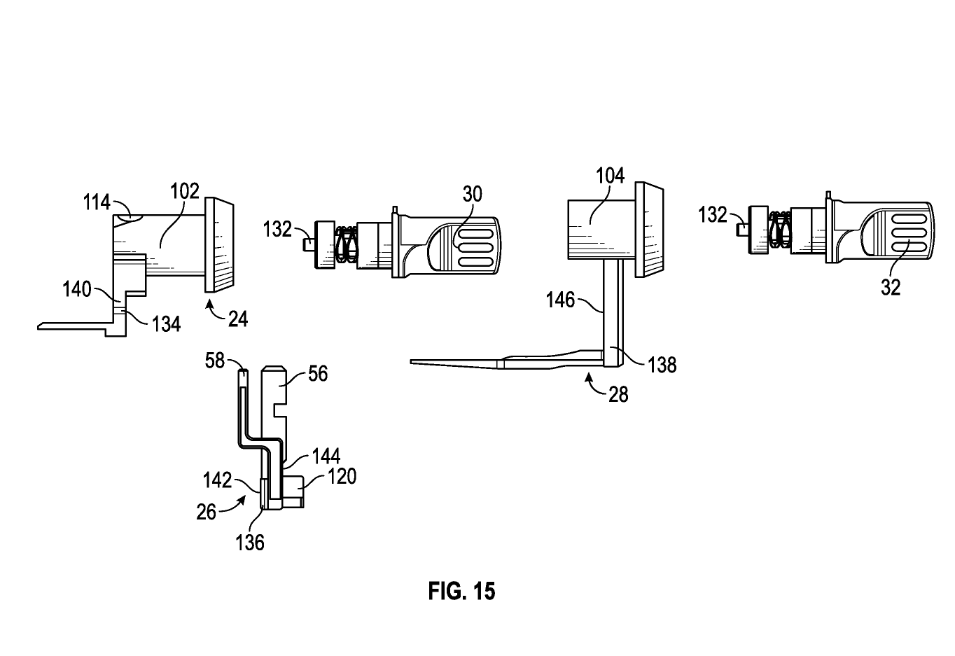

FIG. 14 is an exploded perspective view of adjustable sizer 14 of FIG. 8 showing shim body 24, adjuster body 26 and foot body 28, as well as second adjuster knob 30 and third adjuster knob 32. FIG. 15 is an exploded side view of adjustable sizer 14 of FIG. 8 showing alignment of second adjuster knob 30 with collar 102 in foot body 28, and alignment of third adjuster knob 32 with collar 104 in shim body 24.

Shim body 24 can also include medial shim 48, lateral shim 50, retention tab 110, knob chamber 112 and pin bore 114. Adjuster body 26 can also include slide pin 56, tab 58, pivot point 59, notch 116, slot 118 and stop 120. Foot body 28 can also include medial foot 52, lateral foot 54, pivot hole 126 and knob chamber 128. Second adjuster knob 30 can include finger 130, and third adjuster knob 32 can include finger 132.

Adjuster body 26 can be coupled to shim body 24 such as by inserting slide pin 56 into pin bore 114. Also, notch 134 on shim body 24 can receive tab 136 (FIG. 15) on adjuster body 26. As such, shim body 24 can be configured to translate relative to adjuster body 26 in a longitudinal or linear direction.

Foot body 28 can be coupled to adjuster body 26 such as by inserting pivot point 59 into pivot hole 126. Pivot point 59 can comprise a pair of opposing tabs with flanges that can flex toward each other to allow pivot hole 126 to pass over the flanges. Once foot body 28 is advanced to engagement with adjuster body 26 the tabs can spring back away from each other so that the flanges can prevent the tabs from passing out of pivot hole 126. As such, foot body 28 can be configured to pivot relative to adjuster body 26 and shim body 24 at pivot point 59. Stop 120 can engage wall 138 on foot body 28 to limit the amount of pivoting. As shown, pivot point 59 is centered and one of feet 48 and 50 can be used for a right or left knee. In other examples, pivot point 59 can be offset from the center such that adjustable sizer 14 can be side-specific. For example, a right-side specific device could have pivot point 59 offset closer to foot 54 and a left-side specific device could have pivot point 59 closer to foot 52. In yet other examples, offsetting of pivot point 59 could only move one of feet 48 and 50 relative to its respective shim, leaving the other foot and shim at a consistent distance. This last example could also result in a change in angle from only one condyle.

When fully assembled, face 140 of shim body 24 can abut face 142 of adjuster body 26, and face 144 of adjuster body 26 can abut face 146 of foot body 28.

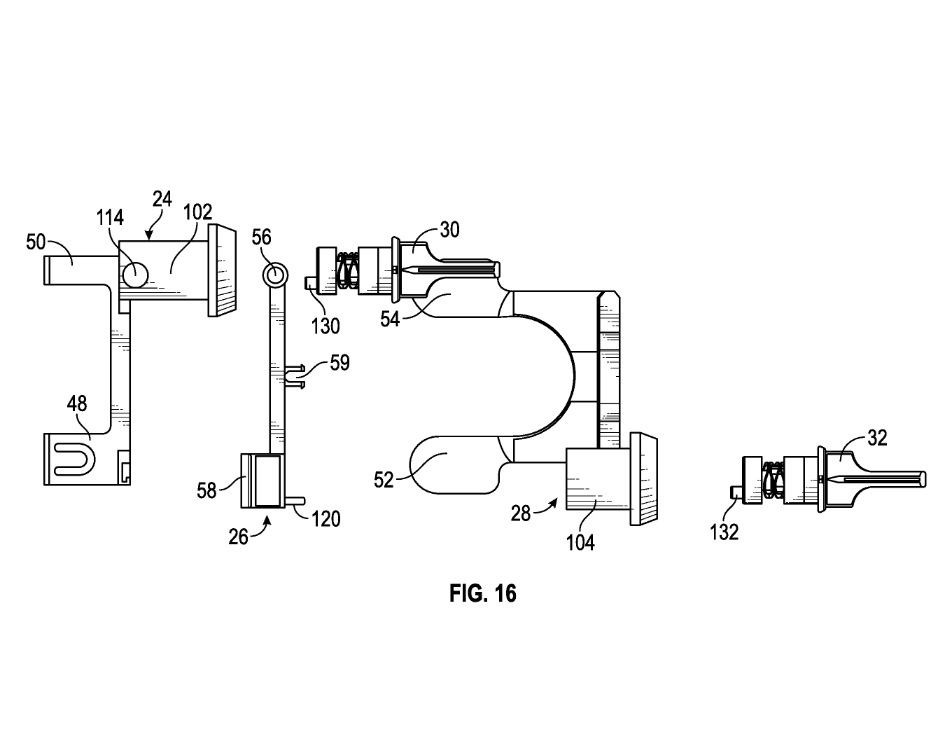

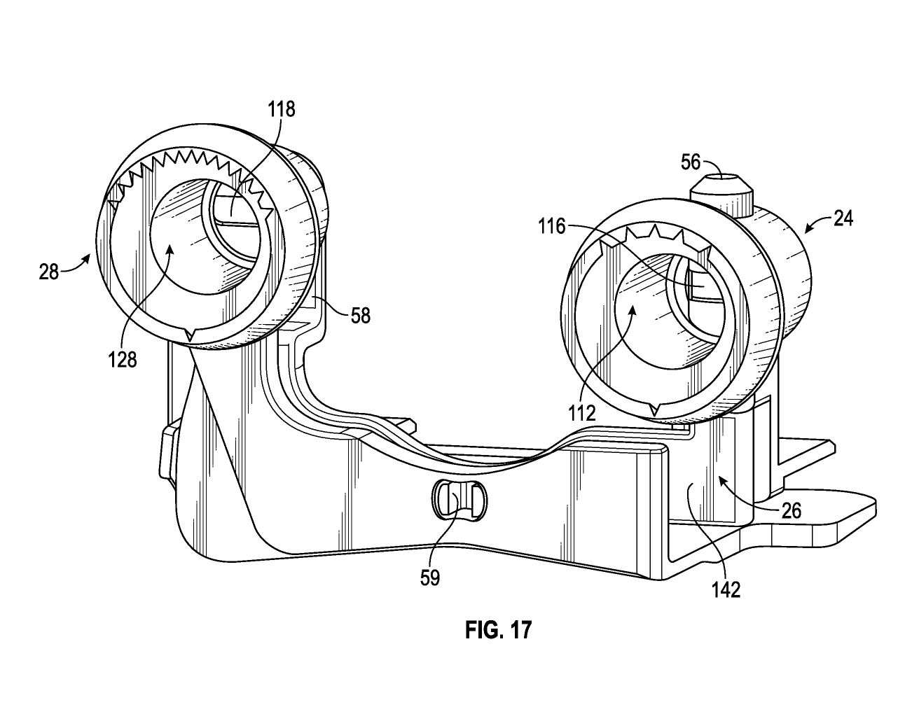

FIG. 16 is an exploded top view of adjustable sizer 14 of FIG. 8 showing second adjuster knob 30 aligned with slide pin 56 of adjuster body 26, and third adjuster knob 32 aligned with slot 118 of adjuster body 26. FIG. 17 is a partial assembled view of adjustable sizer 14 of FIG. 8 with second and third adjuster knobs 30 and 32 omitted to show notch 116 in slide pin 56 within second collar 102, and slot 118 in adjuster body 26 within third collar 104.

Second adjuster knob 30 can be inserted into knob chamber 112 such that finger 130 can be positioned in pin bore 114. As such, finger 130 can engage notch 116 in slide pin 56. As shown in FIG. 17, notch 116 is disposed adjacent knob chamber 112. When adjuster knob 30 is rotated, finger 130 can push against slide pin 56 in notch 116 to move shim body 26 up and down along slide pin 56. Due to confinement of slide pin 56 within pin bore 114, this can cause shims 48 and 50 to move away from or towards feet 52 and 54.

Third adjuster knob 32 can be inserted into knob chamber 128 such that finger 132 can be positioned in pin slot 118 (FIG. 14). As such, finger 132 can engage slot 118 in tab 58. As shown in FIG. 17, slot 118 is disposed adjacent knob chamber 128. When adjuster knob 32 is rotated, finger 132 can push against tab 58 in slot 118 to pivot foot body 28 relative to adjuster body 26 at pivot point 59. Due to the lack of constraint on foot body 28 other than that of pivot point 59, this can cause feet 52 and 54 to be angled relative to shims 48 and 50. For example, foot 52 can move toward shim 48, while foot 54 can move away from shim 50, and vice versa, due to constraint of movement of foot body 28 at pivot point 59.

Referring to FIG. 8, knobs 22, 30 and 32 can be configured to engage with notches in their respective housings in order to lock the knob into a position, which can correspond to a predetermined or known orientation of instrument 10. Chamfer block 20 can include notches 150 that can engage with point 152 of knob 22. Collar 102 can include notches 154 that can engage with point 156 of knob 30. Collar 104 can include notches 158 that can engage with point 160 of knob 32. Notches 150, 154 and 156 can correspond to know dimensions such that the size of the femur can be determined and appropriately sized or prosthetic components can be selected.

4-in-1 cut block 12 and adjustable sizer 14 can be used in conjunction with a surgical plan, either a pre-surgical plan or a plan developed intraoperatively, to size the femur of the patient to receive an artificial knee implant. Once the femur is initially sized, a 4-in-1 block can be selected. 4-in-1 cut block 12 can be selected and configured in different sizes to accommodate different sizes of patient femurs. Either before or after the 4-in-1 block is selected, a distal-most portion of the femur can be removed, including distal portions of the medial and lateral condyles. 4-in-1 cut block 12 can then be sized based on the surgical plan to fit the sized and resected femur by adjusting knob 22.

Adjustable sizer 14 can next be assembled. Adjuster sizer 14 can be pre-set to dimensions from the surgical plan or another setting. If a multi-piece sizer is used, an anterior stylus can be assembled to adjustable sizer 14. For example, a stylus as is described in U.S. Pat. No. 9,050,197 to Lorio et al., which is hereby incorporated by this reference in its entirety for all purposes, can be used with adjustable sizer 14. Next, adjustable sizer 14 can be assembled with the selected 4-in-1 cut block 12.

In one example, the stylus, adjustable sizer 14 and cut block 12 can be assembled together as one unit. In other examples, a surgeon could choose to assemble just two of the components, such as the stylus with adjustable sizer 14 or adjustable sizer 14 and cut block 12. In another example, cut block 12 can be assembled with a fixed (not adjustable) version of a foot. A fixed version of a foot is described in the aforementioned patent to Lorio et al.

Adjustable sizer 14 can be assembled to cut block 12 by insertion of shims 48 and 50 into posterior cut slot 38. In another example, cut block 12 can be provided with an additional slot for receiving shims 48 and 50 such that none of the cut slots are obstructed. For example, a dedicated shim slot could be provided just above or below posterior cut slot 38. Such a dedicated shim slot can be useful with implant systems that may not have consistent posterior or anterior resections and the separate attachment could allow feet 52 and 54 or the stylus to be one part for all the blocks. Next, the assembled components can be placed on the distal femoral resection such that posterior feet 52 and 54 touch the posterior condyles and the anterior stylus references the anterior femoral cortex. In such a position, face 142 of adjuster body 26 can face the resected surface of the femur. The stylus may not fit at this time due to minor differences between the plan and the patient anatomy. The surgeon can decide to vary the components to evaluate fits.

The surgeon can change the size of 4-in-1 cut block 12 by adjusting knob 22, which can alter the gap between anterior cut guide 16 and posterior cut guide 18. The posterior gap between feet 52 and 54 and shims 48 and 50 can be adjusted by rotating knob 32. The posterior angle between feet 52 and 54 and shims 48 and 50 can be adjusted by moving knob 30. As discussed above, points 152, 156 and 160 of knobs 22, 30 and 32 can engage notches 150, 154 and 158, respectively, to dispose instrument 10 into known configurations that correspond to dimensions of various prosthetic components that can be used in the knee replacement procedure. Any one of or any combination of knobs 22, 30 and 32 can be adjusted to evaluate the anterior and posterior gaps and how the femoral cuts relate to the femoral landmarks, like the epicondylar axis, and other bone geometries, like the tibia. This allows the surgeon to evaluate various configurations without removing the components (e.g. cut block 12 and adjustable sizer 14), to fit the patient's anatomy.

Once the size and position is determined, cut block 12 can be pinned to the femur. In one example, pins can be inserted through bores 106 and 108 (FIG. 9) in chamfer block 20. Once pinned, the sizer 14 and the anterior stylus are removed and resections can be made thru slots 36, 38, 40 and 42.

After resections, the pins are removed from chamfer block 20 and 4-in-1 cut block 12 can be removed. This finishes resection of the distal femoral bone in preparation for trialing and any additional steps, such as drilling femoral component lug holes thru the trial femoral component and preparation of the posterior stabilized femoral box, can be taken to complete the procedure.

FIG. 18 shows a distractor instrument 900, which can comprise a tibial arm 904 and a femoral arm 906. The tibial arm 904 can include a plate end 908 and a control end 910. The femoral arm 906 can include a plate end 912 and a control end 914. The arms 904, 906 can be connected to one another at a fulcrum, provided by a pivot pin 916. The pin 916 can extend through aligned holes (not shown) in the arms 904, 906 in an arrangement similar to what might be found in for example a hinge. The tibial arm 904 can be essentially straight, and the femoral arm 906 can be cranked towards the control end. A tibial plate 918 can be provided at the plate end of the tibial arm 908. A femoral plate 920 can be provided at the plate end of the femoral arm 910.

The connection between the arms at the fulcrum can be such that movement of the femoral arm 906 relative to the tibial arm 904 so as to reduce the distance between the control ends of the arms can cause the distance between the plate ends to increase. Similarly, movement of the femoral arm 910 relative to the tibial arm 908 so as to increase the distance between the control ends of the arms can cause the distance between the plate ends to decrease.

The plates are arranged on their respective arms such that the plates can be inserted into the space between the femur F and the tibia T through an anterior incision, and so that the arms 908, 910 extend from the incision in a direction which is generally laterally of the joint. The arms 908, 910 need not extend exactly parallel to the medial lateral axis.

The distractor instrument 900 can be used to distract a knee joint of a patient. This can be achieved by inserting the tibial and femoral plates 908 and 912 into the space between the resected tibia T and the resected femur F while the plate ends of the arms 904, 906 are close together and the control ends of the arms 904, 906 are spaced apart. The joint can be distracted by applying force to the arms to close the space between their control ends, against the force exerted between the control ends of the arms by the spring 932. The displacement of the arms is then locked by means of the ratchet stay 922. Additionally, the distractor instrument 900 can include one or more springs 934 between plates 918 and 920 to perform an alignment check. Furthermore, the distractor instrument 900 can include one or more sensors 936, such as a sensor commercially available from OrthoSensor, Inc. Such a sensor is described in U.S. Pub. No. 2010/0332152 to Stein.

The systems, instruments and methods described herein can greatly minimize the number of implants and the number of instruments needed or required to complete a surgical procedure. Additionally, reduced sets could be available (on the shelf or otherwise) to simplify logistics.

At each step of the procedures described herein, there could be a variety of options available (in surgery or at pre-plan) based on surgeon and patient details and implant and instrument options.

### Various Notes & Examples

Example 1 can include or use subject matter such as a method of planning and preparing for a total knee arthroplasty procedure, the method can comprise: generating three-dimensional models of a tibia and a femur of a patient; sizing the tibia and the femur to within a range based on the three-dimensional models; selecting a resection tool for each of the tibia and femur based on the three-dimensional models; and packaging the resection tools.

Example 2 can include, or can optionally be combined with the subject matter of Example 1, to optionally include femoral and tibial resection tools that are selected from between patient-specific resection tools and a sensor-assisted resection tool.

Example 3 can include, or can optionally be combined with the subject matter of Examples 1 or 2, to optionally include comprising utilizing kinematic data to facilitate resection of the femur.

Example 4 can include, or can optionally be combined with the subject matter of one or any combination of Examples 1 through 3 to optionally include comprising using sensors positioned on the tibia and femur to determine a kinematic axis.

Example 5 can include, or can optionally be combined with the subject matter of one or any combination of Examples 1 through 4 to optionally include utilizing personal data of the patient that includes two or more of height, weight, body mass index, age, gender race, ethnicity, daily activity and disabilities of the patient.

Example 6 can include, or can optionally be combined with the subject matter of one or any combination of Examples 1 through 5 to optionally include using anatomic data including anterior superior iliac spine data.

Example 7 can include, or can optionally be combined with the subject matter of one or any combination of Examples 1 through 6 to optionally include sizing the distal femur after resection using an adjustable contour block.

Example 8 can include, or can optionally be combined with the subject matter of one or any combination of Examples 1 through 7 to optionally include using an adjustable medial/lateral width gage to size the distal femur.

Example 9 can include, or can optionally be combined with the subject matter of one or any combination of Examples 1 through 8 to optionally include comprising using a distractor device to verify alignment of the femur and tibia after resection.

Example 10 can include, or can optionally be combined with the subject matter of one or any combination of Examples 1 through 9 to optionally include selecting one of a tibial resection tool and a femoral resection tool first based on the three-dimensional models and subsequently selecting the other of the tibial resection tool and the femoral resection tool based on a first tool selected.

Example 11 can include, or can optionally be combined with the subject matter of one or any combination of Examples 1 through 10 to optionally include manufacturing at least one of the resection tools as a patient-specific device.

Example 12 can include or use subject matter such as a method of planning and preparing for a surgical procedure, the method comprising: generating a three-dimensional bone model for one or more bones; sizing the one or more bones based on the three-dimensional model; recording a surgical plan based on the three-dimensional bone model; selecting a first surgical tool for the one or more bones based on the surgical plan; and evaluating selection of a second surgical tool based on a performance parameter of the first surgical tool.

Example 13 can include, or can optionally be combined with the subject matter of Example 12, to optionally include packaging the first and second surgical tools.

Example 14 can include, or can optionally be combined with the subject matter of Examples 12 or 13, to optionally include utilizing a computer searchable computer database to select the first surgical tool.

Example 15 can include, or can optionally be combined with the subject matter of Examples 12 through 14, to optionally include selecting the second surgical tool using the computer database based on a list of selectable surgical tools compatible with the first surgical tool selected.

Example 16 can include, or can optionally be combined with the subject matter of Examples 12 through 15, to optionally include a computer searchable database that further comprises anthropometric data.

Example 17 can include, or can optionally be combined with the subject matter of Examples 12 through 16, to optionally include utilizing an adjustable contour block to size the bone.

Example 18 can include, or can optionally be combined with the subject matter of Examples 12 through 17, to optionally include using anatomic data including anterior superior iliac spine data intraoperatively.

Example 19 can include, or can optionally be combined with the subject matter of Examples 12 through 18, to optionally include using sensors positioned on the one or more bones to determine a kinematic axis intraoperatively.

Example 20 can include, or can optionally be combined with the subject matter of Examples 12 through 19, to optionally include further comprising manufacturing at least one of the first and second surgical tools as a patient-specific device.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method of planning and preparing for a total knee arthroplasty procedure, the method comprising:
generating three-dimensional models of a tibia and a femur of a patient;
sizing the tibia and the femur to within a range based on the three-dimensional models;
selecting a resection tool for each of the tibia and femur based on the three-dimensional models; and
packaging the resection tools.

2. The method of claim 1, wherein the femoral and tibial resection tools are selected from between patient-specific resection tools and a sensor-assisted resection tool.

3. The method of claim 2, further comprising utilizing kinematic data to facilitate resection of the femur.

4. The method of claim 3, further comprising using sensors positioned on the tibia and femur to determine a kinematic axis.

5. The method of claim 2, further comprising utilizing personal data of the patient that includes two or more of height, weight, body mass index, age, gender race, ethnicity, daily activity and disabilities of the patient.

6. The method of claim 2, further comprising using anatomic data including anterior superior iliac spine data.

7. The method of claim 2, further comprising sizing the distal femur after resection using an adjustable contour block.

8. The method of claim 7, further comprising using an adjustable medial/lateral width gage to size the distal femur.

9. The method of claim 1, further comprising using a distractor device to verify alignment of the femur and tibia after resection.

10. The method of claim 1, further comprising selecting one of a tibial resection tool and a femoral resection tool first based on the three-dimensional models and subsequently selecting the other of the tibial resection tool and the femoral resection tool based on a first tool selected.

11. The method of claim 1, further comprising manufacturing at least one of the resection tools as a patient-specific device.

12. A method of planning and preparing for a surgical procedure, the method comprising:
generating a three-dimensional bone model for one or more bones;
sizing the one or more bones based on the three-dimensional model;
recording a surgical plan based on the three-dimensional bone model;
selecting a first surgical tool for the one or more bones based on the surgical plan; and
evaluating selection of a second surgical tool based on a performance parameter of the first surgical tool.

13. The method of claim 12, further comprising packaging the first and second surgical tools.

14. The method of claim 12, further comprising utilizing a computer searchable computer database to select the first surgical tool.

15. The method of claim 14, further comprising selecting the second surgical tool using the computer database based on a list of selectable surgical tools compatible with the first surgical tool selected.

16. The method of claim 15, wherein the computer searchable database further comprises anthropometric data.

17. The method of claim 12, further comprising utilizing an adjustable contour block to size the bone.

18. The method of claim 12, further comprising using anatomic data including anterior superior iliac spine data intraoperatively.

19. The method of claim 12, further comprising using sensors positioned on the one or more bones to determine a kinematic axis intraoperatively.

20. The method of claim 12, further comprising manufacturing at least one of the first and second surgical tools as a patient-specific device.
